(19) United States
(12) Patent Application Publication
(10) Pub. No.: US 2009/0018868 A1
(43) Pub. Date: Jan. 15, 2009

(54) Method for Utilizing Cell Therapy Product Facility and Network-Based Business Model Using the Same

(76) Inventors: Jae-Deog Chang, Seoul (KR); Cheong-Ho Chang, Seoul (KR); Dong-Sam Suh, Seoul (KR); Chang-Kwon Ko, Seoul (KR); Seung-Ju Ryu, Gyeonggi-Do (KR); Hee-Chul Ko, Seoul (KR); Sae-Bom Lee, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/224,949

(22) PCT Filed: Mar. 16, 2006
(86) PCT No.: PCT/KR2006/000970

§ 371 Date: Sep. 10, 2008

(30) Foreign Application Priority Data
Mar. 14, 2006 (KR) 10-2006-0023412

(51) Int. Cl. G06Q 50/00; C12N 5/00; G06Q 30/00

(52) U.S. Cl. 705/2; 435/374

(57) ABSTRACT

A method is provided for utilizing a cell therapy product facility and an internet franchise market business comprising: producing a cell therapy product by a CT (Cell Therapy)-module (1) of prefabricated units having specific functions, and; storing of hematopoietic stem cells, bone marrow cells and other cells, by a BC (Banking of Cell and Tissue)-module (2) of prefabricated units having specific functions. Both modules eliminate contamination and maximize efficiency. It provides an operating method and a use method and business method for the above cell therapy product facility. The invention enables easy, low cost production of a product having sufficient quality to be transplanted into patients and put into clinical application within a short period of time. The facility module of the invention can be installed and used wherever adequate space is available. The invention provides improved quality and reliability of cell therapy products.

### BACKGROUND OF THE INVENTION

1. Field of the Invention

The present invention relates to a method for using a cell therapy product facility and a network-based franchise market business method using the same. More specifically, a modularized facility is provided for production of a cell therapy product along with an operating method thereof, and a use method and business method of a medical cell system including: the above cell therapy product facility; a technology for production of a cell therapy product, and a license. Consequently, the present invention enables easy, low cost production of a cell therapy product, of sufficient quality to transplant into patients, within a short period of time, and timely clinical application of the product to patients, via use of the above-mentioned cell therapy product facility.

2. Related Prior Art

As is generally known, cell therapy technology, a next-generation technology, which is expected to bring fundamental changes into well-being trend peculiar to modern societies, and into the public health industry, pharmaceutical industry and medical industry underlying an aging society, is one of the most critical fields for advancement of the medical industry as a technology-intensive and energy-saving industry.

Cell therapy products are medicines used for the treatment, diagnosis and prevention of various diseases by a series of necessary steps involving collecting and proliferating somatic cells from living bodies of patients themselves (autologous) or other people (allogenic) or other animals (xenogenic); or differentiating stem cells into desired cell types, in order to repair impaired or defective cells or tissues and functions thereof. Such cell therapy products have a wide spectrum of application, and over recent years, have received a great deal of attention as a novel therapy having promising and unlimited potential for the treatment of various intractable diseases such as burns, cancers, senile dementia and the like.

Much interest has been directed to cell therapy products as an important 21st century, new drug technology expected to lead the future life science and medical fields, as they have indefinite application fields depending upon the techniques being developed. Several hundred clinical tests and experiments on the cell therapy products are being undertaken in technologically advanced countries including the USA, and a great deal of related research is also being actively undertaken in Korea. Diseases that can be treated by the use of cell therapy products may include, for example, various cancers as well as intractable diseases such as hematologic/immunological disorders and diseases, neurological diseases, diabetes, bone/cartilage diseases and cardiovascular diseases. Further, conquest of intractable diseases via application of stem cells has become the crowning object of the life science world in the 21st century. As a result, there have been technological innovations in medical fields such as cardiovascular systems, nervous systems, blood and immune systems, genetic diseases, hepatic diseases, endocrinal diseases, bone, cartilage and skin diseases and the like.

In recent years, the scientific world and many bio-venture companies have been actively conducting a great deal of research and study on cell therapy products, with some fruitful results, and therefore it is expected that cell therapy products will be spotlighted as the medical industry aims at the world market. Experts in the related art propose that development of cell therapy products will make it possible to treat intractable diseases and substitute for organ transplantation, and therefore will become a next-generation medical industry with an increasing market size.

As such, global interest and necessity for cell therapy products, particularly autologous cell therapy products with secured safety and effectiveness, has increased. However, upon considering the requirement that all factors such as procedures and technologies of manufacturing the cell therapy product with a quality grade transplantable into a patient, and manufacturing facilities should be completely equipped, there is substantially no case in which such cell therapy products are provided by a single system. Therefore, the manufacture of the cell therapy products and the extension of application thereof, are difficult.

### SUMMARY OF THE INVENTION

The present invention has been made in view of the above problems, and it is a first object of the present invention to provide a modularized facility for production of a cell therapy product, an operating method thereof, and a use method and business method for a medical cell system including: the above cell therapy product facility; a technology for production of a cell therapy product; a license; and particularly, a cell therapy product facility, comprising a CT (Cell Therapy)-module capable of producing the cell therapy product and a BC (Banking of Cell and Tissue)-module capable of storing hematopoietic stem cells, bone marrow cells and other cells for a prolonged period of time through appropriate processes.

For this purpose, a second object of the present invention is to provide a facility module for production and storage of a cell therapy product, wherein the CT and BC modules each comprise five functionally specialized units: a preparation unit, a processing unit, a microbial sterility test unit, a quality control unit and a utility unit.

A third object of the present invention is to enable easy, low cost production of a cell therapy product of sufficient quality to transplant into patients within a short period of time and clinical application thereof to patients within an early time, via use of the production facility module.

A fourth object of the present invention is to enable convenient installation and use of such a facility module in any place where a predetermined-size space is available, by provision of the facility module in prefabricated specialized units according to the individual-specific functions.

A fifth object of the present invention is to provide a system capable of producing cell therapy products for hospitals or universities around the world, so that they access treatment technologies using such cell therapy products and thus provide extended opportunity for treating patients.; and at the same time, to provide the above system to the global medical market, thereby accelerating development of cell therapy products.

A sixth object of the present invention is to provide a method for using a cell therapy product facility and a network-based franchise market business method using the same, which enables accomplishment of remarkably improved quality and reliability of the product and thereby enhanced customer satisfaction.

In accordance with an aspect of the present invention, the above and other objects can be accomplished by the provision of a method for using a cell therapy product facility, comprising: producing a cell therapy product by a CT (Cell Therapy)-module composed of separately prefabricated units having individual-specific functions and having an entrance and exit separately partitioned from each other so as to minimize occurrence of contamination; and storing hematopoietic stem cells and bone marrow cells and other cells for a prolonged period of time through appropriate processes, by a BC (Banking of Cell and Tissue)-module composed of a plurality of separately prefabricated units having specific functions and having an entrance and exit separately partitioned from each other so as to minimize occurrence of contamination.

In another aspect of the present invention, there is provided a network-based franchise market business method using a cell therapy product facility, providing access via internet to a management server from a computer connected to the cell therapy product facility transmitting a variety of licenses, authorization business, clinical trial books; supply and sales management and business; education; audio and video data transmission links ; and controlling the cell therapy product facilities CT (Cell Therapy)-module and BC (Banking of Cell and Tissue)-module by the local computer, thereby providing a technology for production of cell therapy products.

### BRIEF DESCRIPTION OF THE DRAWINGS

FIG. 1 is a schematic plan block diagram of a cell therapy product facility CT (Cell Therapy)-module applied to the present invention.

FIG. 2 is a front cross-sectional view of a preparation unit and a utility unit applied to the present invention.

FIG. 3 is a front cross-sectional view of a processing unit and a utility unit applied to the present invention.

FIGS. 4 through 6 are respectively plan, front and side views of an air handling part applied to the present invention.

FIG. 7 is a block diagram showing clean zones of a cell therapy product facility CT (Cell Therapy)-module applied to the present invention.

FIG. 8 is a schematic plan block diagram view of a cell therapy product facility BC (Banking of Cell and Tissue)-module applied to the present invention.

FIG. 9 is a front cross-sectional view of a preparation unit and utility unit of FIG. 8.

FIG. 10 is a block diagram showing clean zones of a cell therapy product facility BC (Baby Cell)-module applied to the present invention.

FIG. 11 is a block diagram of a network-based franchise market business method, using a cell therapy product facility applied to the present invention.

### DETAILED DESCRIPTION OF THE PREFERRED EMBODIMENTS

The preferred embodiments of the present invention for accomplishing the above-mentioned objects will now be described in more detail with reference to the accompanying drawings.

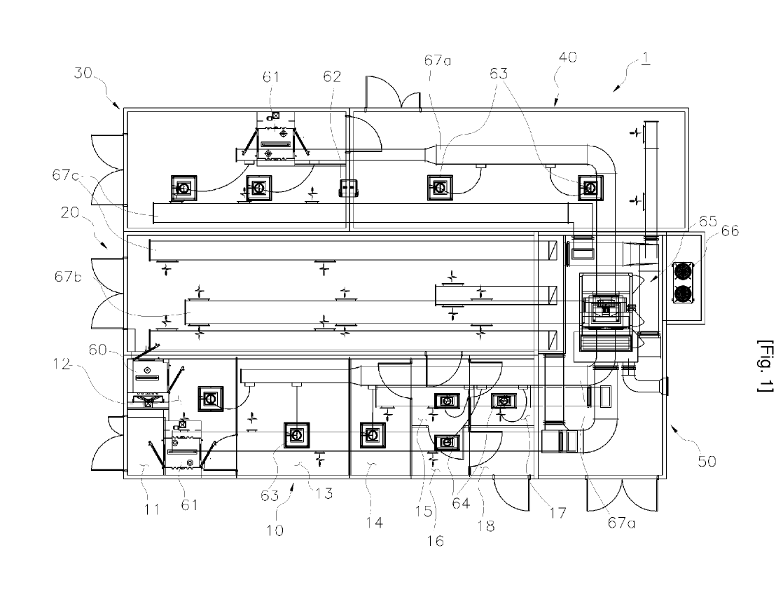

A method for using a cell therapy product facility and a network-based franchise market business method using the same, which are applied to the present invention, are constituted as shown in FIGS. 1 through 11.

In connection with the following description of the present invention, if it is considered that description of known functions or constructions related to the present invention may make the subject matter of the present invention unclear, the detailed description thereof will be omitted.

Terms which will be described hereinafter are established taking into consideration functions in the present invention and may vary according to manufacturer's intention or general practices in the related art. Therefore, the terms used herein should be defined based on the contents of the specification of the present invention.

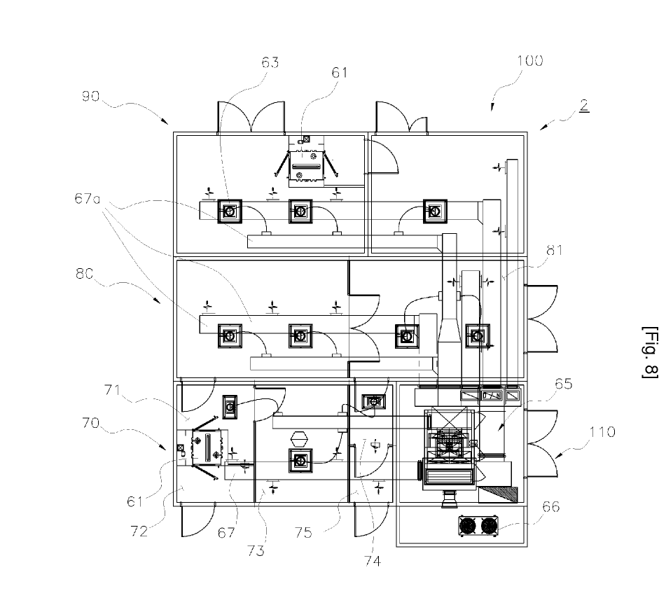

The present invention is directed to a cell therapy product facility, comprising a CT (Cell Therapy)-module 1 (see FIG. 1) including separately prefabricated units having individual functions and separately partitioned entrance and exit so as to minimize occurrence of contamination, and being capable of producing the cell therapy product, and a BC (Banking of Cell and Tissue)-module 2 (see FIG. 8) including separately prefabricated units having individual functions and having partitioned entrance and exit so as to minimize occurrence of contamination, and being capable of storing hematopoietic stem cells, bone marrow cells and other cells for a prolonged period of time through appropriate processes. Each module 1 and 2 is designed to follow a basic layout taking into account a minimal space necessary for processes and optimal size and weight advantageous for transportation.

The technical constitution of the present invention will be described in more detail.

As shown in FIG. 1, the CT (Cell Therapy)-module 1 is provided with a preparation unit 10 for preparing/sterilizing raw materials and storing finished/semi-finished products. Wearing a clean room garment to enter sterile clean zones is required In addition, the CT module 1 includes a processing unit 20 for maintaining the desired level of cleanliness to produce cell therapy products such as cultured chondrocytes and cultured osteoblasts, at the rear of the preparation unit 10.

The facility module of the present invention also includes a microbial sterility test unit 30 for examining probable microbial contamination such as by bacteria during an incubation period for production of cell therapy products, at the rear of the processing unit 20.

At one side of the microbial sterility test unit 30, a quality control unit 40 for confirming safety and effectiveness of the cell therapy products is also provided.

Further, a utility unit 50 for maintenance of essential items such as the desired level of cleanliness, constant temperature and humidity, fire service and electricity for the respective units 10, 20, 30 and 40, is provided at one side of the preparation unit 10.

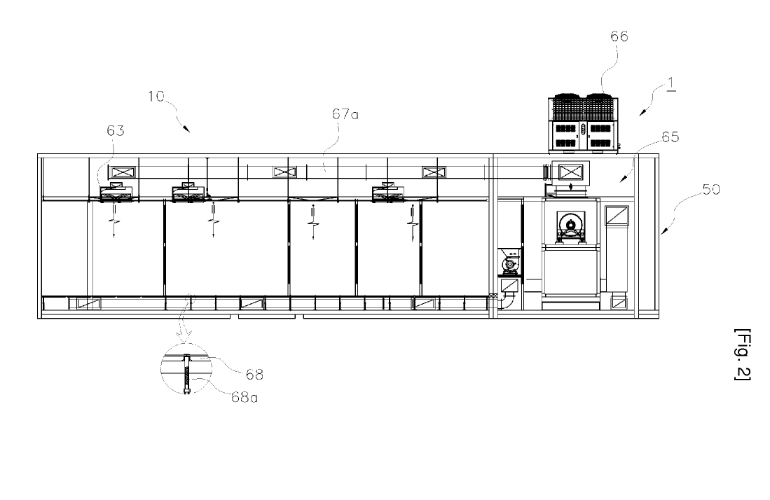

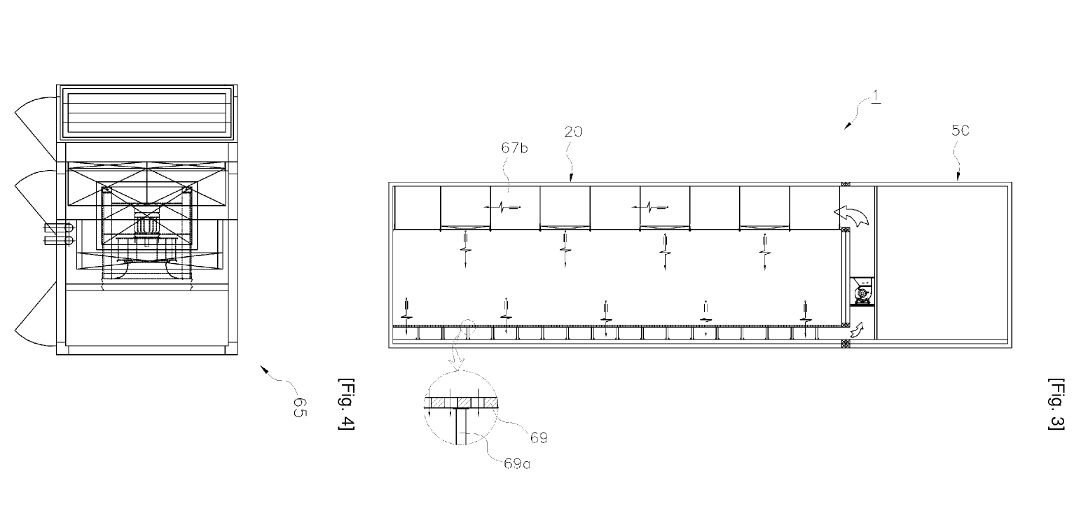

In accordance with the present invention, as shown in FIGS. 2 and 3, the preparation unit 10, processing unit 20, microbial sterility test unit 30 and quality control unit 40 except utility unit 50 are equipped with sterile panels installed at a predetermined height from the bottom, the preparation unit 10, microbial sterility test unit 30 and quality control unit 40 are provided with blank panels 68 at the top of multiple height-adjusting tools 68a arranged at regular intervals, and the processing unit 20 is provided with a grating panel 69 at the top of multiple supporting tools 69a arranged at regular intervals.

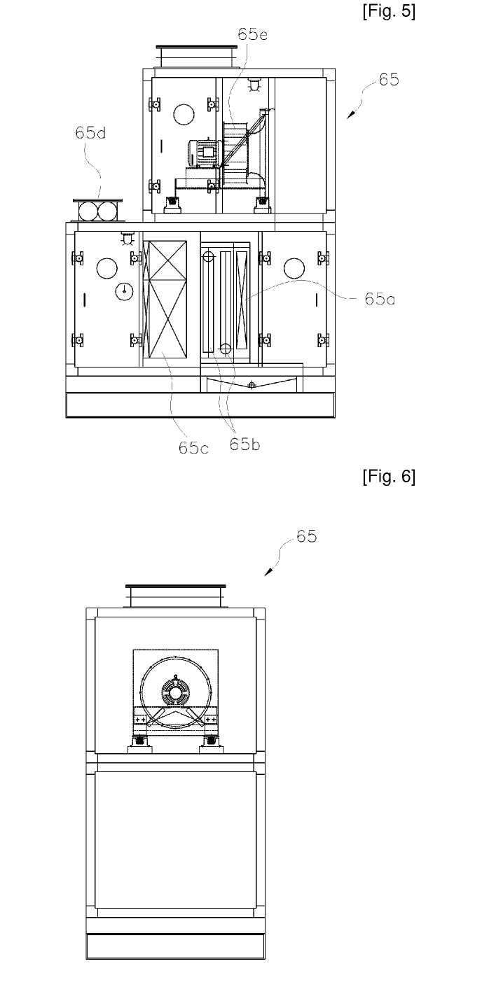

In addition, the present invention includes an air handling part 65 provided inside the utility unit 50 and connected to an air cooler 66, wherein the air handling part 65, as shown in FIGS. 4 through 6, is provided with an air filter 65a for preventing entry of foreign materials, a cooling and heating coil 65b for heat exchange of fluid, a damper 65c for air volume control, a humidifier 65d for water level control, and a fan 65e for air volume control.

The air handling part 65 is connected with a first duct 67a, a passage through which air is allowed to flow through the preparation unit 10, quality control unit 40 and microbial sterility test unit 30. The first duct 67a is provided with first HEPA (High Efficiency Particulate Air) filter units 63 connected thereto at regular intervals, a second duct 67b discharging air to the inside of the processing unit 20, and a third duct 67c for entry of air installed in the respective units 10, 20, 30 and 40. In the third duct 67c, second HEPA filter units 64 are provided at regular intervals.

The inside of the preparation unit 10 is provided with a first dressing room 11 for wearing a first working uniform to enter a washing room or processing unit, a second dressing room 12 for wearing a clean room garment to enter the processing unit, a washing room 13 having an ultrapurification system and providing a space for washing, sterilizing and delivering articles to enter the processing unit, a packaging room 14 for packaging products manufactured in the processing unit, a semi-finished product depository 17 for storing semi-finished products manufactured during processes in liquid nitrogen, a finished product depository 18 for final storage of finished products, packaged and ready for shipment, and first and second buffering zones 15 and 16 for providing clean conditions and serving as buffer areas with the external environment.

In addition, the facility module of the present invention further includes, as shown in FIG. 1, first and second air showers 60 and 61 in the first dressing room 11 of the preparation unit 10, and another second air shower 61 in the microbial sterility test unit 30, whereby contaminating particles from the outside are prevented from entering clean zones and dust or bacteria adhered to the workers are washed and eliminated by high-velocity clean air.

Finally, in accordance with the present invention, between the microbial sterility test unit 30 and quality control unit 40 there is a pass box 62 that enables entrance and exit of articles without personnel entry, thereby preventing escape of contamination source or clean air.

Hereinafter, technical construction of the BC (Banking of Cell and Tissue)-module 2 applied to the present invention will be described in more detail. In this connection, details of technical construction overlapped with those of the CT-module 1 will be omitted herein.

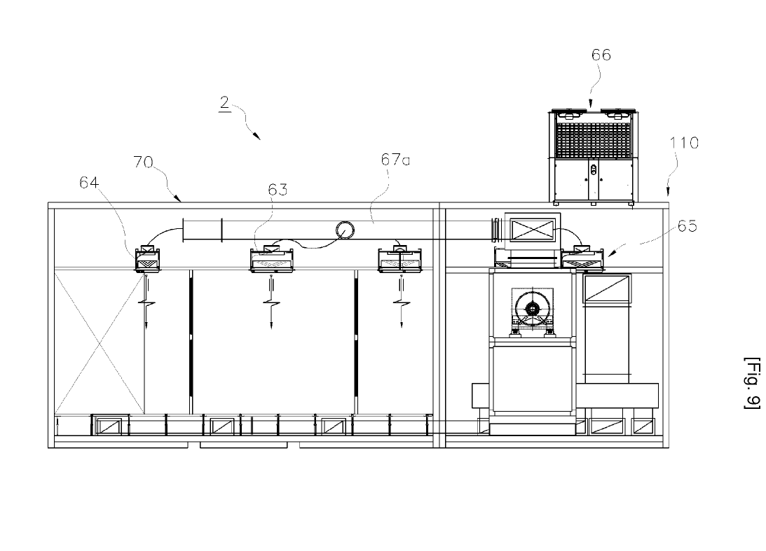

As shown in FIGS. 8 and 9, the BC (Banking of Cell and Tissue)-module 2 is provided with a preparation unit 70 for, while wearing a clean room garment to enter sterile clean zones, preparing/sterilizing raw materials. Here, the preparation unit 70 is provided with a first dressing room 72 for donning a clean room garment (to enter a washing room or processing unit), a washing room 73 including an ultrapurification system and providing a space for washing, sterilizing and delivering articles to enter the processing unit, first and second buffering zones 74 and 75 for providing clean conditions and serving as buffer areas with the external environment, and a head room 71 as a buffer area to enter the processing unit.

In addition, a processing unit 80 for processing and storing umbilical cord material is provided at the rear of the preparation unit 70.

A microbial sterility test unit 90 for examining probable microbial contamination such as by bacteria during transportation or processing of the umbilical cord blood, is also provided at the rear of the processing unit 80.

At one side of the microbial sterility test unit 90, a quality control unit 100 for confirming safety and effectiveness of the cell therapy products is also provided.

Further, at one side of the preparation unit 70, a utility unit 110 is provided for maintenance of essential items such as the desired level of cleanliness, constant temperature and humidity, fire service and electricity for the respective units 70, 80, 90 and 100.

In addition, the BC module of the present invention includes an air handling part 65 provided inside the utility unit 110 and connected to an air cooler 66, a first duct 67a connected to the air handling part 65 through the preparation unit 70, processing unit 80, quality control unit 100 and microbial sterility test unit 90, first and second HEPA filter units 63 and 64 connected at regular intervals to the first duct 67a, a third duct 67c for entry of air provided in the respective units 70, 80, 90 and 100, and second air showers 61 provided in the preparation unit 70 and microbial sterility test unit 90.

A method for using the cell therapy product facility in accordance with the present invention, as constituted above, will be described following.

First, the CT-module 1 for production of the cell therapy product in accordance with the present invention is comprises 5 units, i.e., the preparation unit 10, processing unit 20, microbial sterility test unit 30, quality control unit 40 and utility unit 50. The preparation unit 10 is composed of a dressing room for preparing to enter sterile clean zones, a washing room for preparing and washing raw materials/auxiliary materials used to manufacture products and a depository room for storing finished/semi-finished cell therapy products. The processing unit 20 is the place where cleanliness is kept in class 100 levels and a variety of processes for isolating cells from tissues and differentiating/proliferating cells are carried out. The microbial sterility test unit 30 is a germ-free testing room where cleanliness is kept in class 10000 levels and a sterility test is conducted on raw materials/auxiliary materials before/after processes and final products. The quality control unit 40 is the place where a variety of QC tests except a sterility test are conducted on raw materials/auxiliary materials before/after process and final products. The utility unit 50 is the place where equipment to maintain constant temperature/humidity of the module and a desired level of cleanliness for the respective units is operated. Details thereof will be disclosed following.

The method for using the cell therapy product facility in accordance with the present invention comprises: producing a cell therapy product by a CT (Cell Therapy)-module 1, composed of separate prefabricated units having individual-specific functions, and having an entrance and exit separately partitioned from each other so as to minimize occurrence of contamination; and storing hematopoietic stem cells, bone marrow cells and other cells for a prolonged period of time through appropriate processes by a BC (Banking of Cell and Tissue)-module 2 composed separate prefabricated units having individual-specific functions and having an entrance and exit separately partitioned from each other so as to minimize occurrence of contamination.

The process of producing the cell therapy product includes wearing a clean room garment in a preparation unit 10 in order to enter sterile clean zones, and preparing/sterilizing raw materials and storing finished/semi-finished products therein; maintaining a desired level of cleanliness in a processing unit 20 in order to produce cell therapy products such as cultured chondrocytes and cultured osteoblasts; examining the presence of microbial contamination (such as by bacteria during an incubation period for production) of the cell therapy product, in a microbial sterility test unit 30; confirming safety and effectiveness of the cell therapy product in a quality control unit 40; and maintaining essential items such as a desired level of cleanliness, constant temperature and humidity, fire service and electricity for the respective units (10, 20, 30, 40), in a utility unit 50.

Specifically, in the preparation unit 10, the following processes are carried out: donning a first working uniform in a first dressing room 11 in order to enter a washing room or processing unit; wearing a clean room garment in a second dressing room 12 in order to enter the processing unit; providing an ultrapurification system for washing, sterilizing and delivering articles to the processing unit, in the washing room 13; packaging products from the processing unit in a packaging room 14; storing semi-finished products produced during the manufacturing processes in liquid nitrogen in a semi-finished product depository 17; packaging finished products from the processing unit in the packaging room; storing of finished products until shipment, in a finished product depository 18; and providing clean conditions by first and second buffering zones 15 and 16 as buffer areas with the external environment.

In addition, the first dressing room 11 of the preparation unit is provided with first and second air showers 60 and 61, and the microbial sterility test unit 30 is provided with a second air shower 61, whereby contaminating particles from the outside are prevented from entering clean zones and dust or bacteria adhered to the workers are washed off and eliminated by high-velocity clean air. In addition, a pass box 62 provided between the microbial sterility test unit 30 and quality control unit 40 enables only entrance and exit of articles without personnel entry, thereby preventing escape of contamination sources or clean air.

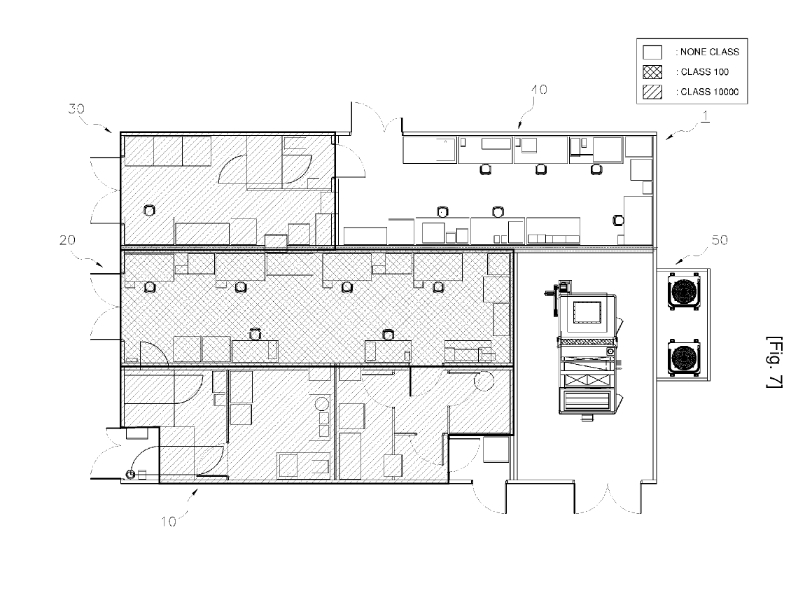

According to the present invention, as shown in FIG. 7, in order to achieve optimal temperature and humidity for cell culture and minimized microbial contamination in the CT-module 1, the processing unit 20 is maintained at a cleanliness level of less than 100 particles having a particle size of 0.5 μm per cubic feet (ft3), the preparation unit 10 and the microbial sterility test unit 30 except the first dressing room and finished product depository are maintained at a cleanliness level of less than 10,000 particles having a particle size of 0.5 μm per cubic feet (ft3), and other areas are divided into zones capable of maintaining cleanliness and differential pressure.

### EXAMPLE 1

In the facility module of the present invention, when an air handling part 65 is active, air is circulated as indicated by arrows through the ducts 67a, 67b and 67c and the grating panel 69. Particularly, where the CT-module 1 is used, preparation of various media and reagents and sterilization of various implements and materials which are necessary for production of cell therapy products, is conducted in the preparation unit 10, and a variety of processes for isolating cells from tissues and differentiating/proliferating cells are conducted in the processing unit 20. For chondrocyte therapeutic, processing of the cell therapy products were carried out in the processing unit 20 of CT-module 1 as follows:

As a first step, cartilage isolation and primary culture were carried out as follows.

1) Biopsy material harvested from hospitals was transferred to the processing unit in the CT module, followed by isolation of cartilage.

2) The biopsy cartilage was cut into small pieces on the sterile workbench, treated with enzymes and placed in a CO2 incubator, followed by isolation of chondrocytes.

3) The chondrocytes were cultured in a culture flask containing a culture medium for about one month.

As a second step, media change and subculture were carried out as follows:

1) For a one-month cell culture period, chondrocytes were allowed to proliferate continuously.

2) Numbers of chondrocytes were proliferated by about 500-fold from initial numbers of 1×105 cells to more than 5×107 cells immediately prior to manufacturing Chondron.

3) During proliferation of chondrocytes, media change was carried out to periodically supply nutrients to cells, and subculture was carried out to facilitate cell proliferation by changing the culture flask.

As a third step, a manufacturing process of chondrocyte therapeutic was carried out: 1) For this purpose, test samples collected before/after processes and from final products were subjected to sterility tests in the microbial sterility test unit (30).
2) Further, except a sterility test, a variety of QC tests such as endotoxin test, mycoplama test using PCR, cell count, cell viability test, virus test, cytotoxicity test and identity test were conducted in the quality control unit 40. Such processes for producing the cell therapy products were collectively carried out in the CT-module 3) After processes and QC tests for the products were complete, the chondrocyte therapeutic was transported to an operating room, followed by chondrocyte transplantation for the treatment of patients with cartilage defects.

The above-mentioned processes were carried out to manufacture chondrocyte therapeutic and bone cell therapy products. However, even though the CT-module 1 is capable of producing chondrocyte therapeutic and bone cell therapy products, the module may also be used to produce other cell therapy products. When production technologies of chondrocyte therapeutic and bone cell therapy products are introduced in conjunction with the CT-module 1, it is possible to perform patient treatment using such cell therapy products and do business associated with treatment of patients.

Similar to the CT-module for production of the cell therapy product, the BC-module 2 for storage of cell therapy product in accordance with the present invention is also comprises five units, i.e., the preparation unit 70, processing unit 80, microbial sterility test unit 90, quality control unit 100 and utility unit 110. The preparation unit 70 is composed of a dressing room for entering sterile clean zones, and a washing room for preparing and washing raw materials/auxiliary materials necessary for manufacturing processes. The processing unit 80 is where cleanliness is kept at class 10000 levels and a variety of processes for isolating cells from tissues or blood and storing cells are carried out. The microbial sterility test unit 90 is a germ-free testing room where cleanliness is kept at class 10000 levels and a sterility test is conducted on raw materials/auxiliary materials before/after processing thereof, and on cells for final storage. The quality control unit 100 is the place where a variety of QC tests except a sterility test are conducted on raw materials/auxiliary materials before/after processing thereof, and on cells for final storage. The utility unit 110 is the place where equipment necessary for maintenance of constant temperature/humidity of the module and cleanliness levels corresponding to the respective units is operated, and details thereof will be disclosed following.

The process, applied to the present invention, of storing hematopoietic stem cells, bone marrow cells and other cells for a prolonged period of time through appropriate processes, includes: donning a clean room garment in a preparation unit 70 in order to enter sterile clean zones, and preparing/sterilizing raw materials therein; storing/preserving the umbilical cord blood in a processing unit 80; examining the presence of microbial contamination, such as by bacteria during transportation or processing of the umbilical cord blood, in a microbial sterility test unit 90; confirming safety and effectiveness of the cell therapy products in a quality control unit 100; and maintaining essential items such as a desired level of cleanliness, constant temperature and humidity, fire service and electricity for the respective units 70, 80, 90 and 100, in a utility unit 110.

Further, in the preparation unit 70, the following processes are carried out including donning a clean room garment in a first dressing room 72 in order to enter a washing room or processing unit; providing an ultrapurification system, and washing, sterilizing and delivering articles to enter the processing unit, in the washing room 73; providing clean conditions by using first and second buffering zones 74 and 75 as buffer areas with external environment; and providing a buffer area to enter the processing unit by a head room 71.

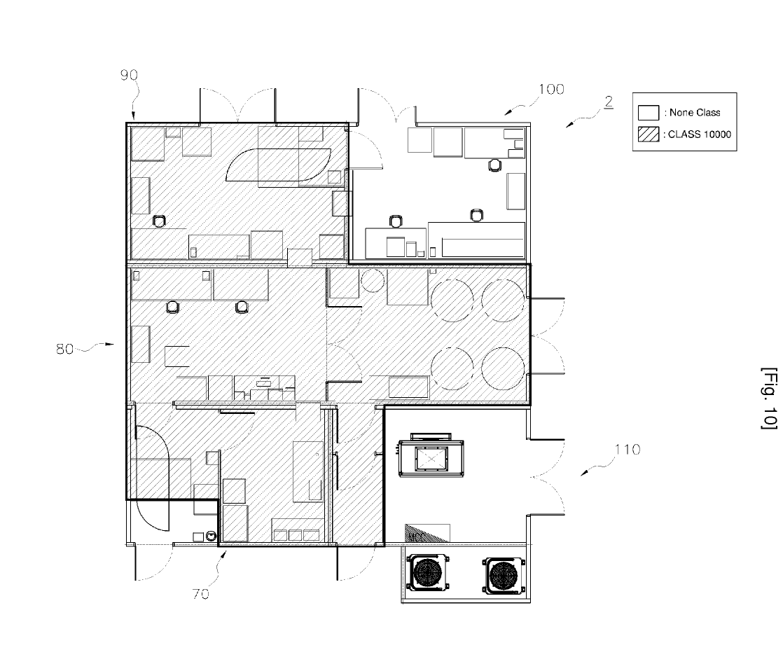

Further, as shown in FIG. 10, in order to achieve optimal temperature and humidity for cell culture and minimized microbial contamination, the preparation unit 70, the processing unit 80 and microbial sterility test unit 90 (except the quality control unit 100, the utility unit 110 and the first dressing room 72) are maintained at a cleanliness level of less than 10000 particles having a particle size of 0.5 μm per cubic feet (ft3). Other areas are divided into zones capable of maintaining a desired level of cleanliness and differential pressure.

The CT-module 1 and BT-module 2 in accordance with the present invention are preferably maintained at a temperature of 22±2° C.C, and humidity of 50±5%.

### EXAMPLE 2

Where the BC-module 2 of the present invention was used, preparation of various media and reagents, and sterilization of various implements and materials which are necessary for cell storage, was conducted in the preparation unit 70, and a variety of processes for isolating cells from tissues or blood and storing cells were conducted in the processing unit 80. For storage of umbilical cord blood-derived hematopoietic stem cells, processing of storage cells was carried out in the processing unit 80 of BT-module 2 as follows.

As a first step, from the umbilical cord blood which was harvested from the umbilical cord, nucleated cells were isolated as follows:

1) A sample was collected from whole blood of the umbilical cord blood harvested from hospitals.

2) Nucleated cells were separated from the sample and were allowed to stand for separation of a red blood cell layer, followed by centrifuging to concentrate a nucleated cell layer.

3) After centrifuging was complete, the top plasma layer was removed using an Auto-Expresser, thereby leaving only a concentrate containing a small amount of the red blood cell layer and a concentrated layer of nucleated cells in the blood unit collection bag.

As a second step, packaging was carried out as follows:

1) The concentrated layer of nucleated cells separated in the first step was transferred to a freezing bag, from which the air was removed.

2) The freezing bag containing the nucleated cell concentrates was placed in a case, followed by sealing.

3) A bar cord was attached to the freezing bag.

4) The freezing bag was packaged to prevent the risk of contamination and was finally inserted into a canister to prepare a finished product.

As a third step, freezing and storage processes were carried out as follows:

1) The finished canister was put into a frame and placed in a freezer equipped with an automatic thermostat.

2) Freezing was initiated.

3) The frozen sample was stored in a liquid nitrogen storage container.

4) Thereafter, in order to demonstrate safety and effectiveness of baby cells, a quality control was carried out as follows:

For this purpose, test samples collected from raw materials/auxiliary materials before/after processing, and cells for final storage were subjected to sterility test in the microbial sterility test unit (90). A variety of QC tests such as cell count, cell viability, hematopoietic stem cell count and colony-forming unit (CFU) assay were also conducted. The above-mentioned processes were carried out to separate and store hematopoietic stem cell from the umbilical cord blood. Therefore, even though the BC-module 2 is capable of separating and storing umbilical cord blood-derived hematopoietic stem cells, such a module may also be used to process and store cell types other than hematopoietic stem cells. When technologies for separation and storage of hematopoietic stem cells from the umbilical cord blood are introduced in conjunction with the BC-module 2, it is possible to do business associated with separation and storage of hematopoietic stem cells.

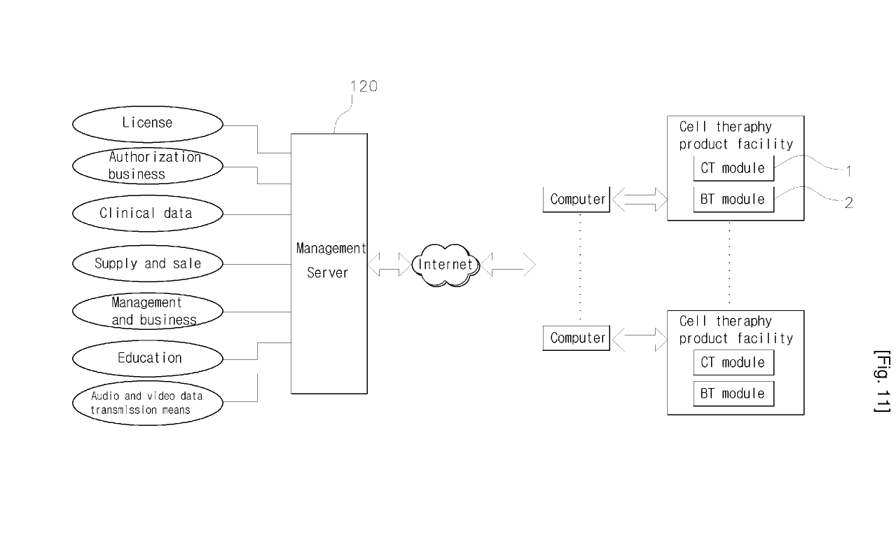

As shown in FIG. 11, the present invention provides a network-based franchise market business method using the cell therapy product facility, comprising: providing a link via internet to a management server 120 to/from a local computer connected to the cell therapy product facility; transmitting a variety of licenses, authorization business, clinical trial book, supply and sales management and business, education, audio and video data transmission means from the management server 120 to the local computer; and controlling the cell therapy product facilities CT (Cell Therapy)-module 1 and BC (Banking of Cell and Tissue)-module 2 by the computer, thereby providing a technology for production of cell therapy product. In particular, the present invention provides procedures and technologies for preparing the cell therapy product having sufficient quality to be transplanted into patients, and a system including all constituents such as a modularized facility. In addition, the present invention supplies a system capable of producing a cell therapy product to hospitals or universities around the world, whereby they obtain treatment technologies using the cell therapy product and thereby provide extended opportunity for treating patients. At the same time, the present invention can provide the technologies of the above system to the global medical market, thereby accelerating development of cell therapy products.

Meanwhile, although the preferred embodiments of the present invention have been disclosed with reference to the accompanying drawings, those skilled in the art will recognize that the present invention may be embodied in different forms with various modifications.

It should be understood that the drawings and detailed descriptions are not intended to limit the invention to the particular form disclosed, but on the contrary, the intention is to cover all modifications, equivalents and alternatives falling within the spirit and scope of the invention as defined by the appended claims.

As apparent from the foregoing, the present invention provides a modularized facility for production of a cell therapy product, operating methods thereof and a use method and business method of a medical cell system including the above cell therapy product facility, a technology for production of a cell therapy product, and a license. In particular, the present invention includes a facility module for production and storage of a cell therapy product, comprising: a CT (Cell Therapy)-module capable of producing a cell therapy product. And; a BC (Banking of Cell and Tissue)-module capable of storing hematopoietic stem cells, bone marrow cells and other cells, for a prolonged period of time through appropriate processes. The CT and BC modules are respectively composed of five functionally specialized units: a preparation unit, a processing unit, a microbial sterility test unit, a quality control unit and a utility unit. The present invention enables easy production at a low production cost of a cell therapy product having sufficient quality to be transplanted into patients within a short period of time, and early clinical application thereof to patients, via use of the above-mentioned facility module. In addition, the present invention enables convenient installation and use of such a facility module in any place where a predetermined-size space is available, by providing the facility module in prefabricated specialized units according to their corresponding functions. Further, the present invention supplies a system capable of producing a cell therapy product to hospitals or universities around the world, whereby they obtain treatment technologies that can be achieved using the cell therapy product, and thereby provide extended opportunity for treating patients. At the same time, the present invention can provide the technologies of the above system to the global medical market, thereby accelerating development of cell therapy products. Consequently, the present invention enables accomplishment of remarkably improved quality and reliability of the product and thereby enhanced customer satisfaction.

What is claimed is:

1-11. (canceled)

12. A method for utilizing a cell therapy product facility, comprising:
producing a cell therapy product by a CT (Cell Therapy)-module (1) composed of separate prefabricated units having specific functions and having an entrance and exit separately partitioned from each other so as to minimize occurrence of contamination; and
storing hematopoietic stem cells and bone marrow cells and other cells for a prolonged period of time through appropriate processes, by a BC (Banking of Cell and Tissue)-module (2) composed of separate prefabricated units having specific functions and having an entrance and exit separately partitioned from each other so as to minimize occurrence of contamination.

13. The method according to claim 12, wherein the process of producing the cell therapy product further comprising:
wearing a clean room garment in a preparation unit (10) in order to enter sterile clean zones, and preparing/sterilizing raw materials and storing finished/semi-finished products therein;
maintaining a desired level of cleanliness in a processing unit (20) in order to produce cell therapy products such as cultured chondrocytes and cultured osteoblasts;
examining the presence of microbial contamination, such as by bacteria, during the incubation period for production of the cell therapy product, in a microbial sterility test unit (30);
confirming safety and effectiveness of the cell therapy product in a quality control unit 40; and
maintaining essential items such as a desired level of cleanliness, constant temperature and humidity, fire service and electricity for the respective units (10, 20, 30, 40), in a utility unit (50).

14. The method according to claim 13, wherein said wearing steps in the preparation unit (10) further comprising steps of:
wearing a first working uniform in a first dressing room (11) in order to enter a washing room or processing unit,
wearing a clean room garment in a second dressing room (12) in order to enter the processing unit,
washing, sterilizing and delivering articles to enter the processing unit and providing an ultrapurification system, in the washing room (13),
packaging products from the processing unit in a packaging room (14),
storing semi-finished products produced during the manufacturing processes in liquid nitrogen in a semi-finished product depository (17),
packaging finished products from the processing unit in the packaging room, followed by final storage of finished products in a finished product depository (18) until shipment, and
providing clean conditions by first and second buffering zones (15, 16) as buffer areas from the external environment.

15. The method according to claim 14, wherein the first dressing room (11) of the preparation unit is provided with first and second air showers (60, 61), and the microbial sterility test unit (30) is provided with a second air shower (61), whereby entrance of contaminating particles from the outside is prevented upon entering clean zones and dust or bacteria adhered to the workers are washed off and eliminated by high-velocity clean air.

16. The method according to claim 13, wherein a pass box (62) provided between the microbial sterility test unit (30) and quality control unit (40) enables only entrance and exit of articles without personnel entry, thereby preventing the escape of contamination sources or clean air.

17. The method according to claim 13, wherein said processing unit (20) is maintained at a cleanliness level of less than 100 particles having a particle size of 0.5 μm per cubic feet (ft3) in order to achieve optimal temperature and humidity for cell culture and minimized microbial contamination, the preparation unit (10) and the microbial sterility test unit (30) (except the first dressing room) and finished product depository are maintained at a cleanliness level of less than 10,000 particles having a particle size of 0.5 μm per cubic feet (ft3), and other areas are divided into zones capable of maintaining a desired level of cleanliness and differential pressure.

18. The method according to claim 17, wherein the temperature is 22±2° C., and humidity is 50±5%.

19. The method according to claim 12, wherein the process of storing hematopoietic stem cells and bone marrow cells and other cells for a prolonged period of time through appropriate processes further comprising:
wearing a clean room garment in a preparation unit (70) in order to enter sterile clean zones and preparing/sterilizing raw materials therein;
storing/preserving the umbilical cord blood in a processing unit (80);
examining the presence of microbial contamination such as by bacteria during transportation or processing of the umbilical cord blood, in a microbial sterility test unit (90);
confirming safety and effectiveness of the cell therapy product in a quality control unit (100); and
maintaining essential items such as a desired level of cleanliness, constant temperature and humidity, fire service and electricity for the respective units (70, 80, 90, 100), in a utility unit (110).

20. The method according to claim 19, wherein said wearing process in the preparation unit (70), the steps further comprising:
wearing a clean room garment in a first dressing room (72) in order to enter a washing room or processing unit;
washing, sterilizing and delivering articles to enter the processing unit and providing an ultrapurification system, in the washing room (73);
providing clean conditions by first and second buffering zones (74, 75) as buffer areas from the external environment; and
providing a buffer area to enter the processing unit by a head room (71).

21. The method according to claim 19, wherein the preparation unit (70), the processing unit (80) and microbial sterility test unit (90) except a quality control unit (100), a utility unit (110) and a first dressing room (72) are maintained at a cleanliness level of less than 10,000 particles having a particle size of 0.5 μm per cubic feet (ft3) in order to achieve optimal temperature and humidity for cell culture and minimized microbial contamination, and other areas are divided into zones capable of maintaining a desired level of cleanliness and differential pressure.

22. The method according to claim 21, wherein the temperature is 22±2° C., and humidity is 50±5%.

23. A network-based franchise market business method using the cell therapy product facility, the process comprising steps of:
providing a source to a management server (120) from a computer connected to the cell therapy product facility via internet;
transmitting a variety of licenses, authorization business, clinical trial book, supply and sales management and business, education, audio and video data from the management server (120) to the computer via internet; and
controlling the cell therapy product facilities CT (Cell Therapy)-module (1) and BC (Banking of Cell and Tissue)-module (2) by the computer, thereby providing a technology for production of cell therapy product.
